(19) United States
(12) Patent Application Publication
(10) Pub. No.: US 2008/0206290 A1
(43) Pub. Date: Aug. 28, 2008

(54) COSMETIC COMBINATION PRODUCT FOR IMPROVING APPEARANCE

(75) Inventors: Jens SCHULZ, Schenefeld (DE); Rainer Kroepke, Schenefeld (DE); Andreas Schepky, Boenningstedt (DE); Julia Eckert, Hamburg (DE); Urte Koop, Hamburg (DE); Sabine Faenger, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/839,384

(22) Filed: Aug. 15, 2007

(30) Foreign Application Priority Data
Feb. 26, 2007 (DE) 10 2007 009 650.1

(51) Int. Cl. A61K 8/02; A61K 31/12; A61K 31/13; A61K 31/195; A61K 31/35; A61K 31/355; A61K 31/415; A61K 31/50; A61P 17/00; A61K 31/522; A61K 31/70; A61K 31/74; A61K 33/26; A61K 33/40; A61K 36/48; A61K 36/82

(52) U.S. Cl. 424/401; 424/617; 424/646; 424/729; 424/757; 424/78.03; 514/249; 514/263.34; 514/385; 514/387; 514/42; 514/456; 514/458; 514/561; 514/564; 514/565; 514/665; 514/678

(57) ABSTRACT

A cosmetic combination product, wherein the product comprises at least two components selected from (a) one or more dietary supplements, (b) one or more cosmetic preparations for topical application to human skin, and (c) one or more patches for adhesion to human skin. Each of the selected components (a) to (c) comprises at least one cosmetically active substance which is also comprised in the other selected component(s). This Abstract is not intended to define the invention disclosed in the specification, nor intended to limit the scope of the invention in any way.

### CROSS-REFERENCE TO RELATED APPLICATIONS

The present application claims priority under 35 U.S.C. § 119 of German Patent Application No. 10 2007 009 650.1, filed Feb. 26, 2007, the disclosure of which is expressly incorporated by reference herein in its entirety.

### BACKGROUND OF THE INVENTION

1. Field of the Invention

The present invention relates to a cosmetic combination product comprising one or more dietary supplements for oral ingestion in combination with one or more cosmetic preparations for topical application and/or with one or more skin patches for application to the skin.

The combination product components, i.e., the dietary supplement(s), the cosmetic preparation(s) and the skin patch(es) comprise ingredients that are coordinated or matched with one another and can be individually selected.

It is thus possible to offer and combine cosmetic skin care or beautification in two or three application forms—externally and/or internally, short-term or permanent or limited in time.

2. Discussion of Background Information

The desire to look beautiful and attractive is deeply engrained in human nature. Even though the ideal of beauty has changed over time, people have always tried to attain a faultless appearance. The condition and appearance of the skin thereby has an important role in a beautiful and attractive appearance.

In order for the skin to be able to fulfill its biological function fully, it needs regular cleansing and care. The cleansing of the skin serves to remove dirt, sweat and the residue of dead skin particles that provide an ideal breeding ground for germs and parasites of all kinds. Skin care products are used mostly for moisturizing and regreasing the skin. Active substances are often added thereto that regenerate the skin and, e.g., are designed to prevent and reduce its premature aging (e.g., the formation of fine lines, wrinkles).

The skin is exposed to constantly changing environmental conditions and over the course of time is also exposed to a number of changes. For example, changes occur in the barrier properties, skin wrinkling and skin elasticity, pigmentation and in particular due to exogenous influences also different inflammatory reactions and, e.g., also to post-reactions of the skin to the impact of UV radiation.

The barrier effect of the skin can be quantified by determining the transepidermal water loss (TEWL). This process involves the evaporation of water from the interior of the body without including the loss of water during perspiration. The determination of the TEWL value has proven to be extremely informative, and may be used for diagnosing cracked or chapped skin, for determining the compatibility of chemically differently composed surfactants and the like.

For the beauty and well-cared appearance of the skin the proportion of water in the uppermost skin layer is of greatest importance. It can be favorably influenced to a limited extent by introducing moisture regulators.

The object of cosmetic skin care is primarily to strengthen or re-establish the natural function of the skin as a barrier against environmental influences, for example, dirt, chemicals, microorganisms, and against the loss of endogenous substances, for example, water, natural fats, electrolytes.

If this function is disturbed, increased resorption of toxic or allergenic substances or attack by microorganisms and as a result thereof toxic or allergic skin reactions can occur.

Cosmetic creams and lotions which have a nurturing effect on the skin have been known for centuries.

Thus, for example, cosmetic preparations which can be used as an anti-cellulite substance are known from DE 10 2005 032237, the entire disclosure whereof is expressly incorporated by reference herein.

Cellulite (medical term: dermopanniculosis) is not a disease but a cosmetic problem. The reasons for cellulite lie above all in the special structure of feminine skin and in the reaction to female hormones. Fat cells are stored in the hypodermis. Their quantity is already determined in infancy and cannot be influenced by nutrition or sport. In the fat cells the fatty acids from nutrition are converted into fats and embedded in the connective tissue in nodal form.

If these fats are not broken down for a longer period (e.g., by exercising), and if the body in addition is overfed, the cells can expand to a multiple of their size. The enlarged cells then press through the connective tissue, and the dreaded orange peel skin occurs, also known as cellulite.

The further consequence in old age are spider veins, varicose veins, thromboses and leg complaints. The upper thighs also often store excess fat that is absorbed via the diet. The unattractive thickening of the upper thighs at the side, also referred to as saddlebags, combined with cellulite are often a great burden on the person afflicted.

For example, the so-called body wrap principle is known for treating cellulite. Anti-cellulite patches (e.g., “Nivea Good by Cellulite Patch® by Beiersdorf) are also known.

WO 05/007127, WO 04/093865, WO 04/074216 and WO 04/060268, the entire disclosures whereof are expressly incorporated by reference herein, likewise disclose numerous cosmetic active substances and treatment methods for treating cellulite.

EP 1181926, EP 728472 and EP 493151, the entire disclosures whereof are expressly incorporated by reference herein, disclose in particular caffeine-containing cosmetics for treating cellulite.

DE 10 2005 053909, the entire disclosure whereof is expressly incorporated by reference herein, describes a self-adhesive skin patch that acts by means of specific active substances in combination with an adhesive composition that is not irritating to the skin as an anti-cellulite patch.

Also dietary supplements as so-called “cosmetics from inside” are known for skin care and skin beautification. For example, dietary supplements which are used, inter alia, to combat the appearance of cellulite are known from WO 2006/023342, EP 1711070, EP 1218001, WO 2006/076240, EP 1729851, EP 0950410, EP 0731699, EP 0579901 and EP 1605764, the entire disclosures whereof are expressly incorporated by reference herein.

With the three known forms of application, dietary supplement, cosmetic preparation and/or skin patch, the user is faced with the problem of being able to combat the individual skin problem in a targeted manner. The user is thereby often overwhelmed in selecting the suitable products due to the different products, different active substances and different directions for use.

Each individual product often acts in only one way and the active substances are not coordinated or matched with one another so that sometimes disappointing results are obtained.

It would therefore be advantageous to have available a cosmetic combination product that helps to solve these problems.

### SUMMARY OF THE INVENTION

The present invention provides a cosmetic combination product which comprises at least two components which are selected from the following components (a) to (c):
(a) one or more dietary supplements,
(b) one or more cosmetic preparations for topical application to human skin, and
(c) one or more patches for adhesion to human skin.
   Each of the selected components (a) to (c) comprises at least one cosmetically active substance which is also comprised in the one or two other selected component(s).

In one aspect, the product may comprise components (a), (b) and (c). In another aspect, it may comprise components (a) and (b), but no component (c), or it may comprise components (a) and (c), but no component (b), or it may comprise components (b) and (c), but no component (a).

In another aspect, component (a) may be present as at least one of a soft gelatin capsule, a hard gelatin capsule, a powder, a granulate, a globule, a lozenge, a tablet, an effervescent tablet, a sucking tablet, a coated tablet, a buccal tablet, a sublingual tablet, a bonbon, a chewing gum, a solution, a tincture, an emulsion, a juice, a concentrate, a syrup and a drinking ampoule. For example, component (a) may comprise a soft gelatin capsule.

In another aspect, component (b) may comprise an emulsion and/or a gel.

In yet another aspect of the product of the present invention, component (c) may comprise a self-adhesive patch.

In a still further aspect of the product of the present invention, the at least one cosmetically active substance may comprise one or more (e.g., one, two, three or four) substances which are selected from carnitine and derivatives thereof (e.g., carnitine tartrate), tea extracts (green tea), mate tea (Ilex paraguariensis), white tea extracts (Camelia sinensis), herb tea extract, ubiquinone Q10, creatine, creatinine, taurine, caffeine, flavonoids, vitamin E, mate tea extract, conjugated linoleic acid (CLA) and derivatives thereof, safflower extract, pantothenic acid, B vitamins, biotin, amino acids and derivatives thereof and folic acid. For example, the at least one cosmetically active substance may comprise one or more (e.g., one, two, three or four) substances which are selected from conjugated linoleic acid (CLA) and derivatives thereof, L-carnitine and derivatives thereof, vitamin E and mate tea extract. In one aspect, the at least one cosmetically active substance comprises L-carnitine and/or a derivative thereof and/or the at least one cosmetically active substance may comprise conjugated linoleic acid (CLA) and/or a derivative thereof, L-carnitine and/or a derivative thereof, vitamin E and mate tea extract.

In yet another aspect, component (a) of the product of the present invention may comprise at least one of soybean oil, lecithin, glycerin, gelatin, titanium dioxide and iron oxide in addition to one or more cosmetically active substances and/or component (b) may comprise at least one substance selected from polyacrylic acid salts, acrylate/C10-30 alkylacrylate crosspolymers and carrageenan in addition to one or more cosmetically active substances.

In another aspect, one or more cosmetically active substances may be comprised in a self-adhesive matrix of component (c) of the product of the present invention. For example, the self-adhesive matrix may comprise a polyacrylate and/or a polyisobutylene in addition to the one or more cosmetically active substances.

In another aspect, the selected components of the combination product may be arranged adjacent to each other. For example, the selected components may be present in a single package.

In another aspect, component (a) may be present in at least one of a blister package, a pan and a dispenser system.

In yet another aspect of the product, the selected components may be used for at least one identical purpose which is selected from a cosmetic treatment and a care and/or beautification of human skin.

The present invention also provides a cosmetic combination product which comprises at least two components which are selected from the following components (a) to (c):
(a) one or more dietary supplements which are present as at least one of a soft gelatin capsule, a hard gelatin capsule, a powder, a granulate, a globule, a lozenge, a tablet, an effervescent tablet, a sucking tablet, a coated tablet, a buccal tablet, a sublingual tablet, a bonbon, a chewing gum, a solution, a tincture, an emulsion, a juice, a concentrate, a syrup and a drinking ampoule,
(b) one or more cosmetic preparations for topical application to human skin which are present as at least one of an emulsion and a gel;
(c) one or more self-adhesive patches for adhesion to human skin.

Each of the selected components (a) to (c) comprises at least one cosmetically active substance which is also comprised in the other selected component(s) and is selected from one or more of carnitine and derivatives thereof (e.g., carnitine tartrate), tea extracts (green tea), mate tea (Ilex paraguariensis), white tea extracts (Camelia sinensis), herb tea extract, ubiquinone Q10, creatine, creatinine, taurine, caffeine, flavonoids, vitamin E, mate tea extract, conjugated linoleic acid (CLA) and derivatives thereof, safflower extract, pantothenic acid, B vitamins, biotin, amino acids and derivatives thereof and folic acid.

In one aspect, the product may comprise components (a), (b) and (c), or the product may comprise components (a) and (b), but no component (c).

In another aspect, component (a) may comprise a soft gelatin capsule.

In yet another aspect, the at least one cosmetically active substance may comprise one or more (e.g., one, two, three or four) substances selected from conjugated linoleic acid (CLA) and derivatives thereof, L-carnitine and derivatives thereof, vitamin E and mate tea extract. For example, the at least one cosmetically active substance may comprise L-carnitine and/or a derivative thereof (e.g. L-carnitine tartrate) or the at least one cosmetically active substance may comprise conjugated linoleic acid (CLA) and/or a derivative thereof, L-carnitine and/or a derivative thereof, vitamin E and mate tea extract.

The present invention also provides a method for the cosmetic prophylaxis or treatment of unevenness in the skin and/or for the treatment of cellulite. The method comprises using the combination product of claim 1.

The present invention also provides a process for making a combination product which is suitable for the care and/or beautification of human skin. The process comprises providing a combination of at least two components selected from the following components (a) to (c):
(a) one or more dietary supplements,
(b) one or more cosmetic preparations for topical application to human skin, and
(c) one or more patches for adhesion to human skin.
   Each of the selected components (a) to (c) comprises at least one cosmetically active substance which is also comprised in the other selected component(s).

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

The combination product or its components, i.e., the dietary supplement(s), the preparation(s) and/or the skin patch(es) comprise one or more of the same active substances. That is, the dietary supplement(s) (a), the preparation(s) (b) and the skin patch(es) (c) contain in each case one or more active substances, at least one of which is also contained in at least one of the other combination product components (a), (b) and/or (c).

Furthermore, the combination product or its components, i.e., the dietary supplement(s), the preparation(s) and/or the skin patch(es) may, for example, all be used for cosmetic treatment, care or beautification of the same type. For example, they may all serve to prevent wrinkles and/or all serve as anti-cellulite agent.

Furthermore, the combination product or its components, i.e., the dietary supplement(s), the preparation(s) and/or the skin patch(es) may, for example, be arranged spatially with respect to one another, such as, e.g., next to one another on a store's shelves or through a combination packaging that includes two or more of the components. The user is thus given a coordinated cosmetic concept without having to search for the matching preparations in different stores, departments, shelves.

It is likewise in accordance with the present invention to coordinate the combination product or its components such that they can be used in a chronologically coordinated manner, e.g., in a specific sequence.

The combination product preferably comprises at least one or more dietary supplements and one or more cosmetic preparations.

In another particularly preferred embodiment, the combination product comprises one or more dietary supplements, one or more cosmetic preparations and one or more (preferably self-adhesive) skin patches.

The user can thus, for example, take (swallow) a dietary supplement (e.g., in the form of a capsule) in the morning, apply the cosmetic preparation to desired areas of the skin to be treated during the day, and apply the corresponding skin patch at night, e.g., while being asleep.

The user thus has available a coordinated concept that makes possible comprehensive skin care. Moreover, incompatibilities can be avoided, since all of the combination product components may contain the same active substance(s) (or at least one identical active substance).

Through this combination the user can obtain a more than satisfactory effect, because he/she is combating the problem as it were day in, day out. The products can also be used according to individual preference, e.g., take a capsule of the dietary supplement twice a day and omit the self-adhesive skin patch, or use the cosmetic preparation (e.g., in the form of a cream) several times a day and use the skin patch while at work and/or being asleep.

The combination product according to the invention is advantageous for this individual manner of application, since the components contain the same active substance(s) (or at least one active substance) and are coordinated (chronologically, spatially) with one another, so that possible side effects, incompatibilities, incorrect application etc., can be reduced to a minimum. Thus also, e.g., due to the tightened security measures when traveling. If the user has to discard the cosmetic preparation in the flight safety zone due to the strict hand luggage rules, he/she is still left with the capsules and the skin patch. This means the treatment does not have to be interrupted.

The advantage of a patch lies, e.g., in the continuous release of active substance(s) over several (e.g., about eight) hours by the patch. Moreover, a patch can also be used at night.

Preferred cosmetic active substances for the dietary supplement(s) can be selected, for example, from one or more of carnitine and derivatives thereof, in particular L-carnitine tartrate, tea extracts (green tea), mate tea (Ilex paraguariensis), white tea extracts (Camelia sinensis), herb tea extract, ubiquinone Q10, creatine, creatinine, taurine, caffeine, flavonoids, vitamin E, mate tea extract, conjugated linoleic acid (CLA), safflower extract, pantothenic acid, B vitamins, biotin, amino acids and derivatives thereof and folic acid.

According to the invention, substances which contribute to skin care and/or beautification when applied topically and/or systematically are regarded as cosmetically active.

Carnitine and derivatives thereof, in particular L-carnitine tartrate, are particularly preferably selected as active substance in two or three of the combination product components.

Carnitine, 3-hydroxy-4-(trimethylammonio)-butyric acid betaine, has the structure

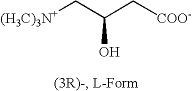

The L-form of carnitine is very widespread in animal tissue and a characteristic constituent of striated musculature, mainly in dark types of meat. L-carnitine is contained only in small amounts (<4 mg/100 g) in vegetable foodstuffs such as fruit, vegetables and grains. The total amount of L-carnitine in the human body is approx. 20-25 g. 98% of the reserves are stored in the myocardium and skeletal muscles.

L-Carnitine acts a carrier molecule in the transport of long-chain fatty acids through the inner mitochondrion membrane into the mitochondrial matrix area, while medium and short-chain fatty acids can also pass through it without an esterification with L-carnitine.

L-Carnitine is offered in numerous products also as dietary supplement. The target groups are (endurance) athletes and overweight persons to whom L-carnitine is offered to improve performance or as a weight-loss agent (“fat-burner”). The efficacy is very controversial in both cases. Since an L-carnitine deficiency is very rare in healthy people, no benefit is to be anticipated from a carnitine supplement. Carnitine is not consumed in its biochemical function as a carrier, so that an increase in consumption in the area of lipometabolism does not lead to an increased need for carnitine. Conversely, an additional intake of carnitine does not lead to an increase in fatty acid oxidation. An excess of carnitine is secreted via the kidneys.

In the case of cardiovascular disease, L-carnitine, through an increase in the β-oxidation of the fatty acids, increased ATP level, a reduction in the in the blood counts and tissue fat values (free fatty acids) and through an increase in the blood circulation through the heart, can improve the cardiac output and overall increase the cardiac capacity. Moreover, a certain immunostimulation function is accredited to L-carnitine that is attributed to an increase in the activity of the granulocytes, t-lymphocytes and killer cells.

It has surprisingly proven that a self-adhesive matrix containing carnitine and a cosmetic preparation containing L-carnitine that can be applied topically have a positive effect on the reduction of cellulite. The lymphatic circulation is stimulated.

The combination product according to the invention containing carnitine is therefore suitable for the care of those areas of the skin which are affected by cellulite. The preferred embodiments of the present invention therefore include the use of the combination product for the treatment of skin conditions caused by cellulite.

The active substance L-carnitine (preferably as the tartrate) is preferably contained in at least two of the combination product components, for example, in all three thereof, i.e., dietary supplement, cosmetic preparation and skin patch.

Safflower extract (safflower oil) is an extract or oil with a high content of linoleic acid. This polyunsaturated fatty acid prevents the skin from drying out.

The conjugated linoleic acids (abbreviated as CLA, CAS no. 2420-56-6, formula: C₁₈H₃₂O₂) are a fatty acid group with eight geometric isomers, certain isomers of which are found in particular in meat and dairy products from ruminants.

There are several different CLA isomers: the 9,11 isomer (cis-9, trans-11—intermediate product of the biological hydrogenation of polyunsaturated fatty acids in the rumina of ruminants) contributes in particular to the growth of muscles. The 10,12 isomer prevents lipogenesis (storage of fat in the fat cells). Available CLA products generally contain both isomers in equal proportions.

Studies in humans prove that CLA reduces the body fat ratio, while at the same time the, ratio of muscle is increased.

Linoleic acid is particularly preferred, obtained from the oil of the safflower or other vegetable oils or derivatives thereof, in particular as triglyceride.

The mate plant (Ilex paraguariensis) grows wild as brush in the Araucaria forests of Brazil, Uruguay and Paraguay, on the highlands between the Paraná, Paraguay and Uruguay rivers and their feeder rivers, thus its home is the Paraná basin jungle. Even today, however, about half of the mate on the market is harvested from trees growing wild.

Mate is a popular infused drink that is obtained by infusing chopped dried leaves of the Ilex paraguariensis. Mate was originally used to refer to the drinking vessel (from the Quechua word mati, drinking vessel), today also to refer to the drink that was called caiguá by the Guarani. However, the leaves are referred to as yerba (Spanish for herb). Nowadays, mate is often associated with the “gaucho tradition,” but in fact it was being drunk before the colonization of the original inhabitants of South America.

Originally, and now once again in the more northern, warmer areas of southern South America, the brew is drunk cold ad terere (very refreshing in summer).

In South America sometimes other infusion drinks are also referred to as mate, e.g., mate de coca, prepared from the leaves of the cocoa bush.

The fresh leaves contain about 0.35 to 1.7% of caffeine, approx. 0.1 to 0.2% of theobromine, theophylline and 4 to 16% of tannins. The infusion is yellowish green and depending on the strength contains caffeine, theobromine, chlorophyll, tannic acid, etheric oil and vanillin. As far as vitamins are concerned, it contains vitamins A, B1, B2 and C. The aroma of green and roasted mate was separated by gas chromatography and analyzed by mass spectrometry, and about 200 of more than 250 components were identified thereby. In descending order ketones, aldehydes and aliphatic alcohols, heterocyclic compounds, carboxylic acids and lactones, terpene alcohols, furanones, phenols, aromatic compounds, pyrazines and pyrroles were detected. Pyrroles, pyrazines and furanones occur above all in the Maillard reaction during the roasting process.

Pantothenic acid belongs to the vitamins of the B complex. It has the structural formula:

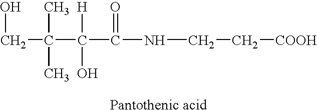

It is readily water-soluble and heat-sensitive. In foodstuffs pantothenic acid is found mostly in bonded form as a constituent of coenzyme A. It cannot be stored in the body, must therefore be absorbed daily with food. Pantothenic acid is mainly involved in enzymatic reactions in metabolic processes. In cell metabolism it is used for the production of energy. Furthermore, it contributes to the composition of different neurotransmitters, fatty acids, carbohydrates, cholesterol, hemoglobin and vitamins A and D.

The recommended intake to avoid deficiency symptoms is currently 6 mg per day. Pantothenic acid is present in many vegetable and animal foodstuffs. Liver, wholewheat products and eggs have a high content.

Since virtually every foodstuff contains pantothenic acid, a deficiency is very rare. However, intestinal disease, such as colitis ulcerosa, and alcohol addiction can lead to a deficiency. Dialysis patients and diabetics can also possibly experience a deficiency of this vitamin.

In addition to or instead of the preferred carnitine and/or CLA, other suitable active substances for the purposes of the invention can be added to the combination product components either individually or in combination, in particular active substances that have a positive impact on the condition of the skin. It has thus been shown that active substances for a positive impact on old skin, which reduce the occurrence of wrinkles or even of existing wrinkles include, for example, biochinones, in particular ubiquinone Q10, isoflavone and isoflavonoids, genistein, arctiin, cardiolipin, lipoic acid, anti freezing proteins, hop extracts and hop/malt extracts, substances that promote the restructuring of the connective tissue, isoflavonoids and isoflavonoid-containing plant extracts such as, e.g., soybean and clover extracts, which can also be used very well in the cosmetic preparations and skin patch matrices for use according to the invention. The combination product according to the present invention also is particularly suitable for using active ingredients for aiding the skin functions in dry skin, such as, for example, vitamin C, biotin, creatine, creatinine, propionic acid, glycerin, green tea extracts or solutions, and white tea extracts or solutions.

In a similar way, the incorporation of active ingredients for alleviating or positively influencing irritative skin conditions, whether for sensitive skin in general or for skin irritated by noxae (UV light, chemicals) is advantageous. Mention may be made here, for example, of active ingredients such as sericosides, various extracts of licorice, licochalcone A, silymarin, silyphos, flavonoids, dexpanthenol, ethanol, inhibitors of prostaglandin metabolism, in particular of cyclooxygenase and of leukotriene metabolism, in particular of 5-lipoxygenase, but also of the 5-lipoxygenase inhibitor protein, FLAP.

The incorporation of pigmentation modulators also is advantageous. Mention may be made here, for example, of active ingredients which reduce the pigmentation of the skin and thus lead to a cosmetically desired lightening of the skin and/or reduce the appearance of age spots or lighten existing age spots, such as tyrosine sulfate, Vitamin C, lipoic acid and liponamide, various extracts of licorice, kojic acid, hydroquinone, arbutin, fruit acids, in particular alpha-hydroxy acids (AHAs), bearberry (Uvae ursi), ursolic acid, ascorbic acid, green tea extracts, aminoguanidine and pyridoxamine.

In the same way the dietary supplements, cosmetic preparations or matrices for use according to the invention are an excellent basis for active substances that cause an intensified/more rapid tanning of the skin (Advanced Glycation End products (AGE), with or without the influence of UV light, lipofuscins, nucleic acid oligonucleotides, purines and pyrimidines and NO-releasing substances.

The use of mate or green tea extract is preferred, since in combination with carnitine a skin-care effect and above all a cellulite reducing effect can be observed.

The secret of white tea is due to its gentle processing, so that it remains virtually unchanged. White tea extracts contain a high content of polyphenols, which are among the highly effective antioxidants that render free radicals harmless. Research dealing with these aspects of white tea is still relatively young. White teas are commercially available under the names Yin Zhen (silver needle) and Yin Long (silver dragon).

The use of white tea extract is therefore also preferred since in combination with carnitine a skin-care effect and above all a cellulite reducing effect may be observed.

The use of mate tea, coenzyme Q10, warming substances and panthenol is also preferred. At any rate, the combination product of the present invention can also be used to achieve a regular appearance of the skin.

Dietary supplements are concentrates of nutrients or other substances with nutritionally specific or physiological effect, which are introduced in a dosed form and are intended to supplement general nutrition. According to the invention, in particular dietary supplements or the active substances contained therein are selected such that a cosmetic effect, i.e., the care or beautification of the skin can be observed.

In addition to the above-mentioned active substances, furthermore the following preferred active substances can also be selected from carotenoides, catechines, isoflavones from soybeans and red clover; calcium, zinc, selenium, iron, magnesium, silicon, silica, algae, guarana, cranberry, cacao extracts, collagen, hyaluronic acids, vitamin C, vitamin A, fatty acids, such as, e.g., omega-3 fatty acids, omega-6 fatty acids, gamma-linoleic acid, unsaturated vegetable oils, taurine, fiber, roseroot, asparagus extract, cinnamon extracts, ginger, proteins, flavonoids, saponins, herb extracts, vegetable extracts and/or fruit extracts.

In addition to the selection according to the invention of at least one active substance that is contained in two or more components of the combination product, one skilled in the art can select the active substances cited above as preferred. Naturally, he/she also selects the active substances according to their compatibility, so that, e.g., different substances can be contained additionally in dietary supplements than in cosmetic preparations or patches.

One, two or preferably all of the substances chosen from conjugated linoleic acid (CLA) and derivatives thereof, L-carnitine and derivatives thereof, in particular L-carnitine tartrate, vitamin E and mate tea extract may be used as active substances in the combination product components.

The following substances can be contained in the dietary supplements as so-called auxiliaries, which one skilled in the art can choose freely according to form of use, mode of action and application method.

Starch derivatives (corn, wheat, potato starch), cellulose derivatives (hydroxyl propyl cellulose, methyl cellulose, fructose, glucose, mannitol, sorbitol, ethyl cellulose, sodium carboxymethylcellulose), lactose, vegetable oils, soybean oil, neutral oil, vegetable and synthetic mono-di and triglycerides, waxes and/or lipids are preferably chosen as fillers/carriers.

Dry binders, polyethylene glycols, polyvinyl pyrrolidone, calcium hydrogen phosphate, PVP (collidone) and/or gelatin are preferably chosen as binders.

Magnesium stearate, calcium behenate, glycerol monostearate, vegetable fats, highly disperse silicon dioxide (Aerosil), lecithin, calcium carbonate, calcium phosphate, magnesium phosphate, mannitol, cellulose, salts of the nutrient fatty acids, magnesium salts of the nutrient fatty acids, thermoxidized soybean oil esterified with mono and diglycerides, potassium carbonate, magnesium carbonate, magnesium hydroxide, magnesium oxide, sodium ferrocyanide, potassium ferrocyanide, calcium ferrocyanide, calcium silicate, magnesium silicate, talc, aluminum silicates, fatty acids, beeswax, candelilla wax and carnauba wax and/or microcrystalline wax are preferably chosen as lubricant/flow regulation agent/mold release agent.

The following substances are particulary suitable as colorants:

Yellow: Curcumin; riboflavin; phosphate ester; tartrazine; quinoline yellow
Orange: Yellow orange S
Red: Carmine; azorubin; amaranth; cochineal red A; erythrosine; red 2G; allurared AC
Blue: Patent blue V; indigotin; brilliant blue FCF
Green: Chlorophyll; cupreous complexes of chlorophylls and chlorophyllins; Green S
Yellow, orange and red: Carotene; annatto; capsanthin; lycopin; beta-apo-8′-carotenal; E 160f-beta-apo-8′-carotenal ethylester; lutein; canthaxanthin; zeaxanthin; betanin; anthocyane
White, red, metals: calcium carbonate; E 171—titanium dioxide; E 172 iron oxide; E 173—aluminum; E 174—silver; E 175—gold.

Lecithin, propylene glycol alginate, polysorbate 20, polysorbate 80, polysorbate 40, polysorbate 60, polysorbate 65, ammonium phosphatides, hydroxypropylcellulose, hydroxypropyl methylcellulose, salts of the nutrient fatty acids, mono and diglycerides of nutrient fatty acids, acetates of mono and diglycerides of fatty acids, lactates of mono and diglycerides of fatty acids, citric acid esters of mono and diglycerides of fatty acids, tartaric acid esters of mono and diglycerides of fatty acids, diacetyltartaric acid esters of mono and diglycerides of fatty acids, acetate-tartrate esters of mono and diglycerides of fatty acids, sugar esters of nutrient fatty acids, sugar glycerides, polyglycerin esters of nutrient fatty acids, polyglycerin-polyricinoleate, propylene glycol esters of nutrient fatty acids, thermoxidized soybean oil esterified with mono and diglycerides, sodium stearoyl-2-lactylate, calcium stearoyl-2-lactylate, stearyl tartrate, sorbitan monostearate, sorbitan tristearate, sorbitan monolaurate, sorbitan monooleate, sorbitan monopalmitate, hydroxypropyl starch, hydroxypropyl distarch phosphate and/or sodium starch octenylsuccinate are preferably chosen as emulsifiers.

Alginic acid, alginates, agar agar, carrageen, starch and starch derivatives (e.g., monostarch phosphate, phosphatized distarch phosphate, acetylated distarch phosphate, acetylated starch, acetylated distarch adipate, hydroxypropyl starch, hydroxylpropyl distarch phosphate, sodium starch octenylsuccinate) carob bean flour, guar flour, tragacanth, gum arabic, xanthan; karaya gum, tara gum, gellan gum, sorbitol, mannitol, glycerin, konjak root, pectin, cellulose and cellulose derivatives (e.g., methyl cellulose, hydroxypropylcellulose, hydroxypropylmethylcellulose, methylethyl cellulose, sodium carboxymethylcellulose, crosslinked carboxymethyl cellulose), salts of the nutrient fatty acids, e.g., magnesium salts of the nutrient fatty acids, acetates of mono and diglycerides of fatty acids, fatty acids, beeswax, candelilla wax, carnauba wax, shellac, microcrystalline wax and hydrous poly-1-decene are preferably chosen as gelling agents, thickeners and humectants.

Sorbitol, mannitol, aspartame, cyclamate, isomalt, saccharin, sucralose, thaumatin, neohesperidin DC, maltitol, lactitol, xylitol, fructose, sucrose, lactose, glucose, artificial and synthetic aromas and/or aroma preparations are preferably chosen as sweetening agents/aromas.

The dietary supplement according to the invention is preferably present in the form of a capsule, in particular in the form of a soft gelatin capsule.

Other forms of administration are preferably hard gelatin capsules, powders, granulates, globules, lozenges, tablets, effervescent tablets, sucking tablets, coated tablets, buccal tablets, chewable tablets, sublingual tablets, bonbons, chewing gums, solutions, tinctures, emulsions, juices, concentrates, syrup and/or drinking ampoules.

The corresponding packaging or seals, blisters, tubes etc. are known from the prior art.

The dietary supplement is preferably present in a portioned form for a single dose such as, e.g., as a capsule, coated tablet or as a powder. The capsules or coated tablets may, for example, be sealed in a blister pack. In powder form, the supplement can be packaged, e.g., in little paper envelopes.

Other forms, liquid, as bonbon (hard shell, soft core), as a tablet, etc., are also conceivable and according to the invention. The type and form are known from the prior art.

The dietary supplement is preferably packaged in blisters, pans or dispenser systems. A package unit preferably comprises a one-month or two-month supply (30-60 capsules).

In addition to the above-mentioned active substances, in particular conjugated linoleic acid, carnitine and/or mate tea extract are preferably included. Particularly preferably all three substances are comprised in the dietary supplement. Advantageously, vitamin E and/or lecithin are also present.

According to the invention, in this case the cosmetic preparation then advantageously comprises, e.g., carnitine (preferably as tartrate) only, carnitine (tartrate) and other cosmetic active substances, such as, e.g., folic acid or Q10, carnitine (tartrate) and vitamin E, or also all three of carnitine (tartrate), mate tea extract and vitamin E.

It is naturally preferred according to the invention if all three components, i.e., the dietary supplement, the cosmetic preparation and the skin patch, comprise, e.g., carnitine (tartrate) and vitamin E.

The (preferably capsular) dietary supplement comprises one or more active substances, at least one of which, preferably all of which, active substances are also contained in the other combination product components, i.e., the cosmetic preparation and/or the skin patch. Furthermore, the dietary supplement preferably comprises as base materials soybean oil, lecithin, glycerin, gelatin, titanium dioxide and iron oxide.

By way of non-limiting example, one or more dietary supplements may comprise the ingredients shown in Table 1 below.

TABLE 1
Preferred ingredients of the dietary supplement (a)

| Function | Constituent | Commercial Name | Manufacturer/Supplier |
|---|---|---|---|
| Active | Conjugated | Tonalin ® TG 80 | Cognis |
| substance | linoleic acid | | |
| | 200-250 mg | | |
| Active | L-carnitine | Carnipure ® tartrate | Lonza |
| substance | | micronized | |
| Active | Mate fol extr. | EFLA ® 920 | Frutarom Switzerland |
| substance | S. siccum | | Ltd. |
| | 30-50 mg | | |
| Active | Vitamin E | e.g., D, L-alpha | e.g., Cognis, DSM |
| substance | 5-15 IU | Tocopherol USP (E | |
| | | 307) | |
| Auxiliary | Lecithin | Sternlecithin B-10 | Solae Europe, S.A. |
| | (E 322) | Special | |
| | 0.5-200 mg | | |

Table 1 also discloses the preferred quantities of the ingredients and from whom they can be ordered.

TABLE 2
Exemplary Formula 1 - Filling composition without gelatin casing

| Active substances of dietary supplement | W (mg) |
|---|---|
| Conjugated linoleic acid | 200 |
| Vitamin E | 5 |
| L-carnitine (as tartrate) | 40 |
| Green mate leaf powder extract | 40 |
| Lecithin from soybean, E 322 | 5.000 |
| Ingredients dietary supplement | W (mg) |

Exemplary Formula 2 - Dietary supplement with soft gelatin shell

| | |
|---|---|
| Conjugated linoleic acid, triglyceride | 300.000 |
| RRR-alpha tocopherol concentrate | 20.000 |
| L-carnitine tartrate | 80.000 |
| Green mate leaf powder extract | 100.000 |
| Soybean oil, hydrogenated, Ph.Eur | 30.795 |
| Lecithin from soybean, E 322 | 7.000 |
| Glycerol 85%, Ph.Eur. | 60.00 |
| Gelatin, 200 Bloom, Ph.Eur.,NF | 115.00 |
| Titanium dioxide, Ph.Eur., USP, E 171, CI No. 77891 | 2.04 |
| Red iron oxide, E 172, CI no. 77491, NF | 1.02 |

Exemplary Formula 3 - Dietary supplement with soft gelatin shell

| | |
|---|---|
| Conjugated linoleic acid, triglyceride | 200.000 |
| RRR-alpha tocopherol concentrate | 5.225 |
| L-carnitine tartrate | 43.980 |
| Green mate leaf powder extract | 40.000 |
| Soybean oil, hydrogenated, Ph.Eur. | 30.795 |
| Lecithin from soybean, E 322 | 5.000 |
| Glycerol 85%, Ph.Eur. | 58.73 |
| Gelatin, 200 Bloom, Ph.Eur., NF | 114.90 |
| Titanium dioxide, Ph.Eur., USP, E 171, CI no. 77891 | 2.04 |
| Red iron oxide, B 172, CI no. 77491, NF | 0.51 |

Exemplary Formula 4 - Dietary supplement with soft gelatin shell

| | |
|---|---|
| Conjugated linoleic acid, triglyceride | 100.00 |
| RRR-alpha tocopherol concentrate | 2.220 |
| L-carnitine tartrate | 20.000 |
| Green mate leaf powder extract | 1.000 |
| Soybean oil, hydrogenated, Ph.Eur. | 30.795 |
| Lecithin from soybean, E 322 | 2.000 |
| Glycerol 85%, Ph.Eur. | 58.73 |
| Gelatin, 200 Bloom, Ph.Eur., NF | 114.90 |
| Titanium dioxide, Ph.Eur., USP, E 171, CI no. 77891 | 4.04 |
| Red iron oxide, E 172, CI no. 77491, NF | 0.20 |

The preferred quantities per dietary supplement are
CLA (conjugated linoleic acid), daily dose about 10 mg-2 g, about preferably 50 mg-1 g, particularly preferably about 200-600 mg
Vitamin E, daily dose: about 2 mg-800 mg, preferably about 2.5 mg-200 g, particularly preferably about 5-20 mg
L-carnitine, daily dose: about 5 mg-3 g, preferably about 10 mg-200 mg, particularly preferably about 10-100 mg
Lecithin, corresponding to a choline content of about <500 mg

The preferred recommended dose is about 1-10 capsules, the daily intake of about 1-3 capsules is particularly preferred.

The cosmetic preparation according to the invention can preferably be composed according to the disclosure of DE 10 2005 032237, the entire disclosure whereof is expressly incorporated by reference herein.

Preferably the cosmetic preparation according to the invention as a combination product component comprises one or more active substances, at least one of which, preferably all of which, active substances are also contained in the other combination product components, i.e., the dietary supplement(s) and/or the skin patch(es).

The active substance(s) of the cosmetic preparation are advantageously present in a total concentration of from about 0.0001% to about 12.5% by weight and preferably in a total concentration of from about 0.01% to about 0.8% by weight, in each case based on the total weight of the preparation.

Furthermore, the cosmetic preparation advantageously comprises polyacrylic acid salts, acrylate/C10-30 alkylacrylate crosspolymers and/or carrageenan as base materials.

An example of a polyacrylic acid salt which is preferred for use in the present invention is sodium polyacrylate (CAS: 9003-04-7), which is available, e.g., from Cognis under the trade name COSMEDIA SP. These polyacrylic acid salts are not crosslinked.

It is advantageous according to the invention to use carbomers as crosslinked polyacrylic acid salts. An example of a crosslinked polyacrylic acid salt which is preferred for use in the present invention is sodium carbomer (e.g., CAS: 9007-16-3, CAS: 9007-17-4, CAS: 9062-04-8, CAS: 76050-42-5), which is available, e.g., from Noveon under the trade name Carbopol 980.

An example of an acrylate/C10-30 alkylacrylate crosspolymer (INCI: acrylates/C10-30 alkylacrylates crosspolymer) which is preferred for use in the present invention is available, e.g., from Noveon under the trade name Pemulen TR-1.

An example of a carrageenan which is preferred for use in the present invention is filed under the CAS No.: 9000-07-1 (EINECS No.: 232-524-2) and can be purchased, e.g., from Interorgana under the trade name Gelcarin GP-379NF.

Embodiments of the cosmetic preparation according to the present invention that are particularly advantageous comprise as further components from about 0.01% to about 2.5% by weight of vinylpyrrolidone/vinylacetate copolymer (VP/VA copolymer), based on the total weight of the cosmetic preparation.

An example of a vinylpyrrolidone/vinylacetate copolymer (VP/VA copolymer) which is preferred for use in the present invention is a polymer that is composed of about 60% of vinylpyrrolidone and about 40% of vinylacetate and is available, e.g., under the trade name Luviskol VA64W from BASF.

The cosmetic preparation according to the invention advantageously shows a weight ratio of polyacrylic acid salts to acrylates/C10-30 alkylacrylates crosspolymer of from about 1:10 to about 10:1 and preferably from about 1:1 to about 2:1.

The cosmetic preparation according to the invention advantageously shows a weight ratio of polyacrylic acid salts to carrageenan of from about 1:10 to about 10:1.

The cosmetic preparation according to the invention advantageously shows a weight ratio of polyacrylic acid salts to vinylpyrrolidone/vinylacetate copolymers of from about 1:10 to about 10:1 and preferably from about 1:1 to about 2:1.

Examples of the cosmetic preparations which are suitable for use in the present invention include preparations which comprise the emulsifier combination of polyacrylic acid salts, crosslinked polyacrylic acid salts, acrylate/C10-30 alkylacrylate crosspolymer and carrageenan in a total concentration of from about 0.05% to about 9.5% by weight and preferably in a concentration of from about 0.5 to about 1.8% by weight, in each case based on the total weight of the cosmetic preparation.

The cosmetic preparation preferably is an aqueous or aqueous-alcoholic gel or gel cream, referred to herein in a simplified manner as a “gel”.

Gel creams are particularly light products with a low emulsifier and lipid content. They are characterized in that the can be easily distributed over the skin and impart a refreshing feeling. After the product has been applied, little or no residue should remain on the skin. Gel creams generally contain a relatively high proportion of hydrophilic thickeners. Since the thickener or the thickener system is in the external phase, it has a significant impact on the sensory properties of the product. Conventional thickener systems either cannot be distributed, do not impart a refreshing feeling or leave behind an excessively sticky residue on the skin.

The aqueous phase of the cosmetic (gel) preparations according to the invention may advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and/or active substances such as self-tanning agents, insect repellents vitamins, plant extracts, niacinamide, panthenol.

It is preferred for the cosmetic preparation according to the invention to comprise as further constituents alpha-hydroxy acids and/or salts thereof. Lactic acid/lactates or citric acid/citrates are thereby preferably used according to the invention, for example, in a concentration of from about 0.01% to about 5% by weight, based on the total weight of the preparation.

Advantageously the preparation according to the invention may comprise other cosmetic additives, such as the UV sunscreen filters and preservatives or preservative auxiliaries. Active substances of this type can be advantageously contained in the preparation according to the invention in concentrations from, e.g., about 0.01% to about 30% by weight, based on the total weight of the preparation.

Advantageously the cosmetic preparation according to the invention comprises at least one water-soluble colorant.

Advantageously the cosmetic preparation according to the invention has a transparent or translucent appearance. Advantageously, it may be stored in a transparent packaging.

It also is advantageous if the cosmetic preparation according to the invention is free from surfactants with an HLB value of greater than about 25. It is preferred for the cosmetic preparation according to the invention to be free from surfactants with an HLB value of greater than about 20. The HLB values of surfactants can be taken from the customary tabular compilations (e.g., Fiedler, Lexikon der Hilfsstoffe für Pharmazie, Kosmetik und angrenzende Gebiete, Editio Cantor Verlag, Aulendorf, the entire disclosure whereof is expressly incorporated by reference herein).

An emulsion of a cosmetic preparation (preferably of an aqueous or aqueous-alcoholic gel) and the skin's endogenous lipids is also in accordance with the present invention. An emulsion of this type is produced in that the cosmetic preparation (preferably of an aqueous and aqueous-alcoholic gel or gel cream) is applied to the skin and subsequently rubbed in.

The method for producing an emulsion according to the invention of this type, wherein a cosmetic preparation (preferably of an aqueous or aqueous-alcoholic gel or gel cream) according to the invention is applied to the skin and subsequently rubbed in, is also in accordance with the present invention. Rubbing the cosmetic preparation into the skin is thereby preferably carried out with the hands or fingers, but can also be carried out by means of an applicator (e.g., a sponge or a massage head).

The preparation can be advantageously present, e.g., in the form of a thin, sprayable aqueous or aqueous-alcoholic solution, in the form of a gel, as an ointment, cream or lotion (optionally sprayable). The preparation is preferably present as a gel.

The preparation can also be used advantageously as a spray or impregnating medium for a patch or cloth. Patches and cloths impregnated with the preparation according to the invention are therefore also in accordance with the present invention, a transition to the combination product component skin patch being given here, as long as self-adhesion is provided.

The (preferably self-adhesive) skin patch according to the invention preferably comprises a matrix which adheres to human skin and at least one cosmetic active substance, the active substance preferably being contained in the matrix.

According to the invention, the one or more active substance(s) contained in the matrix of the skin patch are also contained in one or more of the other combination product components, i.e., the dietary supplement(s) and/or the cosmetic preparation(s).

The skin patch according to the invention may have a structure as disclosed in DE 10 2005 053909, the entire disclosure whereof is expressly incorporated by reference herein.

The skin patch according to the invention encompasses all patches for cosmetic application, such as, e.g., patches, pads, cloths, plasters, dressings, poultices, bandages, masks.

It is preferred according to the invention for a cosmetically active substance, not a medically active substance, to be contained in the adhesive matrix.

Polymers, polyisobutylenes or poultices which form a gel in water are preferred as a self-adhesive matrix. In particular an adhesive mass based on polyacrylic acid and/or polyacrylates is preferred.

The proportion of polymers which form a gel in water such as, e.g., polyacrylic acid in the matrix regulates the adhesive capacity. In particular the matrices disclosed in DE 10260873 and DE 10056010, the entire disclosures whereof are expressly incorporated by reference herein, are particularly advantageous for use in the present invention.

Advantageous polyacrylates for use in the present invention include acrylate-alkyl acrylate copolymers, in particular those chosen from the so-called carbomers or carbopols (Carbopol® is a registered trademark of B.F. Goodrich Company). The acrylate-alkyl acrylate copolymer(s) that are particularly advantageous according to the invention are characterized by the following structure:

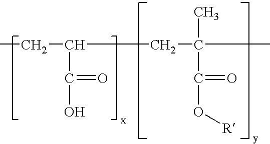

In this structure R′ is an alkyl radical, in particular a long-chain alkyl radical, and x and y are numbers which indicate the respective stoichiometric fraction of the respective comonomers.

Acrylate copolymers and/or acrylate-alkyl acrylate copolymers that are particularly preferred in accordance with the invention are those available under the commercial designations Carbopol® 1382, Carbopol® 981 and Carbopol® 5984 from the B. F Goodrich Company, preferably polyacrylates from the group of the Carbopols of types 980, 981, 1382, 2984 and 5984, and more preferably Carbomer 2001.

Advantageous are further copolymers of C10-30 alkyl acrylates and one or more monomers of acrylic acid, of methacrylic acid and esters thereof, which are crosslinked with an allyl ether of sucrose or with an allyl ether of pentaerythritol.

The polymer which forms a gel in water, especially polyacrylic acid and/or copolymers thereof, is used preferably in an amount of about 2-55% by weight, particularly preferably about 5-30% by weight.

The polymer matrices are preferably prepared without the use of organic solvents, preferably at about 40-95° C., in standard commercial mixers/compounders or, continuously, in suitable extruders.

A further suitable polymer which forms a gel in water is, inter alia, baobab flour.

For example, the combination of polymer (polyacrylic acid) which forms a gel in water, seaweed extract, such as alginates and/or agar agar, and a monohydric or polyhydric alcohol has proven to be advantageous. In this way it is possible, using water, polymer which gels in water, sea algae extract, and monohydric or polyhydric alcohol as starting materials, to produce, in a targeted fashion, soft, smooth, self-adhesive hydrogel matrices as a basis for production and use as patches, TTS, cataplasms or cosmetic pads/matrices.

In order to obtain particular performance properties it is possible for the polymer matrices to be admixed with appropriate plasticizers, solubilizers, penetration enhancers, neutralizing agents such as tromethamol (2-amino-2-(hydroxymethyl)-1,3-propanediol), triethanolamine (2,2′,2″-nitrilotriethanol) or NaOH, for example, fillers and/or other known additives, although it is not mandatory to add them.

In one embodiment which is particularly preferred in accordance with the invention the polymer matrix or gel matrix comprises active dermatological or cosmetic substances for controlled local and/or systemic delivery onto/into the skin, in amounts of up to about 35% by weight, preferably up to about 15% by weight, in particular up to about 2% by weight.

Since the matrix of the invention may be a water-containing application form, a cooling effect is obtained in addition, this effect already per se being cosmetically pleasant and contributing to well-being. This positive effect can be intensified by the addition of further care constituents. Besides glycerin it is possible in particular to add serinol (3-amino-1,2-propanediol) and/or isoserinol (2-amino-1,3-propanediol) and also urea and PCA (pyrrolidone carboxylic acid) as moisturizers. It is of course also possible to add further substances for this purpose.

Polyisobutylene PIB is also preferably used as a matrix system according to the invention.

In addition to PIB polyisobutylene, hydrophobic base polymers such as SIS (styrene-isoprene-styrene) triblock copolymers, SBS (styrene-butadiene-styrene) triblock copolymers, SBR (copolymers of styrene and butadiene), synthetic and/or natural polyisoprene, polyamide, polyester, copolyester, polyurethane and/or mixtures thereof are non-limiting examples of possible further matrix materials, alone or in combination. Out of the plurality of known polymer matrices, polyacrylates and polyisobutylenes are particularly preferred.

As a matrix base, polyisobutylenes meet the requirements particularly well for a self-adhesive polymer matrix that is gentle on the skin and can be removed in a pain-free manner, so that it is logical to preferably select polyisobutylene as a matrix base.

SBR is a collective term for copolymers of styrene and butadiene, which contain both monomers generally in a weight ratio of about 23.5:76.5, in exceptional cases also about 40:60, and the macromolecules of which predominantly comprise the structural units I and II:

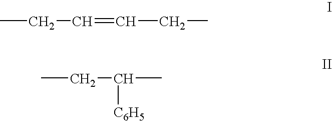

Water-containing matrices according to the invention can be used to supply very dry areas of the skin with moisture.

The polymer matrix according to the invention as a plaster, pad or skin patch is thus extremely suitable for skin care purposes and in particular, for simple cooling purposes and furthermore is easy to use, since it is self-adhesive.

It is also advantageous for the polymer matrix to be solvent-free, to thereby avoid disadvantages of the prior art.

The combination product according to the invention which preferably comprises at least one dietary supplement and at least one cosmetic preparation and optionally also a self-adhesive skin patch, is particularly predestined for cosmetic prophylaxis or cosmetic treatment of unevenness in the skin and/or cellulite.

The use of the combination product components to produce a combination product for the care and/or beautification of human skin is likewise preferred.

The cosmetic prophylaxis or cosmetic treatment of cellulite according to the invention can be carried out advantageously in combination with massage equipment, ultrasound, infrared light, textiles, bandages and/or occlusive foils.

The advantage and the therefore preferred use of the combination product lies in that all the components can be used for the same cosmetic treatment, care and/or beautification of human skin. That is, all the components may be used, for example, for treating skin conditions caused by cellulite.

### EXAMPLES

### Combination Product Component—Cosmetic Preparation in the Form of a Gel

| | 1 | 2 | 3 | 4 | 5 |
|---|---|---|---|---|---|
| Polyacrylic acid, Na salt | 1.0 | 0.2 | 0.5 | 0.1 | 1.5 |
| (carbomer) | | | | | |
| PEG-40 stearate | 0.5 | — | 0.75 | — | — |
| Carrageenan | 1.25 | 0.2 | 0.2 | 0.1 | 2.5 |
| Sodium polyacrylate | 0.2 | 0.2 | 0.3 | 0.5 | 1.5 |
| Vinylpyrrolidone/vinyl acetate | 1.0 | — | 0.2 | — | — |
| copolymers | | | | | |
| Acrylates/C10-C30 alkyl | 1.5 | 0.2 | 0.15 | 1.5 | 0.75 |
| acrylate crosspolymer | | | | | |
| Water-soluble dye | 0.01 | 0.4 | — | — | — |
| Cyclomethicone | 1 | 8.5 | 5 | 0.5 | 3 |
| Dimethiconol | 0.5 | 0.5 | 0.75 | 1.25 | 1.0 |
| Cetylstearyl alcohol | — | — | 1 | 1 | — |
| Hydrous polyisobutene | 0.5 | — | 1.0 | — | 0.25 |
| Carnitine | 0.5 | 0.1 | 0.75 | 3.0 | 0.25 |
| Panthenol | 0.5 | — | 0.75 | 0.25 | 0.1 |
| Glycerin | 3.0 | 8.6 | 12.5 | 17.2 | 5.0 |
| Perfume | q.s. | q.s. | q.s. | q.s. | q.s. |
| Methylpropanediol | 4.5 | 2.0 | 0.5 | 1.5 | — |
| Propylparaben | 0.3 | — | 0.25 | 0.15 | — |
| Ethanol | — | 10.0 | 5.0 | — | 3.5 |
| Water | ad | ad | ad | ad | ad |
| | 100 | 100 | 100 | 100 | 100 |

The exemplary dietary supplements listed in Table 2 by way of example, in combination with a cosmetic preparation according to Example 3, produce an advantageous combination product that can be used to combat the visible skin changes through cellulite.

For example, the gel can be applied to the affected areas of the skin daily after showering and the dietary supplement can be taken after a meal, e.g., breakfast.

A chronologically coordinated sequence of the application of the combination product components is to be stipulated according to the invention. The user can thus for example apply the affected areas of the skin with the gel preparation according to the invention in the morning after rising, swallow the capsule after breakfast and apply a corresponding skin patch at night.

The skin patch is particularly preferably present as a flat embodiment with a total area of from about 0.2 to about 1000 cm², preferably in an area of from about 8 to about 15 cm to about 10 to 20 cm. Large areas (up to about 1000 cm²) may thus be covered, for example, on the upper thigh for the treatment of cellulite.

The skin patch preferably has a polymer matrix weight content of from about 0.1 to about 1000 g, in particular about 14 g per skin patch. The shape of the skin patch can, for example, be round, oval, angular or adapted to the areas of the skin to which it is to be applied.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A cosmetic combination product, wherein the product comprises at least two components selected from components (a) to (c):
(a) one or more dietary supplements,
(b) one or more cosmetic preparations for topical application to human skin, and
(c) one or more patches for adhesion to human skin,
and wherein each of the selected components (a) to (c) comprises at least one cosmetically active substance which is also comprised in the other selected component(s).

2. The product of claim 1, wherein the product comprises components (a), (b) and (c).

3. The product of claim 1, wherein the product comprises components (a) and (b), but no component (c).

4. The product of claim 1, wherein the product comprises components (a) and (c), but no component (b).

5. The product of claim 1, wherein the product comprises components (b) and (c), but no component (a).

6. The product of claim 1, wherein component (a) is present as at least one of a soft gelatin capsule, a hard gelatin capsule, a powder, a granulate, a globule, a lozenge, a tablet, an effervescent tablet, a sucking tablet, a coated tablet, a buccal tablet, a sublingual tablet, a bonbon, a chewing gum, a solution, a tincture, an emulsion, a juice, a concentrate, a syrup and a drinking ampoule.

7. The product of claim 6, wherein component (a) comprises a soft gelatin capsule.

8. The product of claim 1, wherein component (b) comprises at least one of an emulsion and a gel.

9. The product of claim 1, wherein component (c) comprises a self-adhesive patch.

10. The product of claim 1, wherein the at least one cosmetically active substance comprises one or more substances selected from carnitine and derivatives thereof, tea extracts (green tea), mate tea (Ilex paraguariensis), white tea extracts (Camelia sinensis), herb tea extract, ubiquinone Q10, creatine, creatinine, taurine, caffeine, flavonoids, vitamin E, mate tea extract, conjugated linoleic acid (CLA) and derivatives thereof, safflower extract, pantothenic acid, B vitamins, biotin, amino acids and derivatives thereof and folic acid.

11. The product of claim 10, wherein the at least one cosmetically active substance comprises one or more substances selected from conjugated linoleic acid (CLA) and derivatives thereof, L-carnitine and derivatives thereof, vitamin E and mate tea extract.

12. The product of claim 11, wherein the at least one cosmetically active substance comprises at least one of L-carnitine and a derivative thereof.

13. The product of claim 11, wherein the at least one cosmetically active substance comprises at least one of conjugated linoleic acid (CLA) and a derivative thereof, at least one of L-carnitine and a derivative thereof, vitamin E and mate tea extract.

14. The product of claim 1, wherein component (a) comprises at least one of soybean oil, lecithin, glycerin, gelatin, titanium dioxide and iron oxide in addition to one or more cosmetically active substances.

15. The product of claim 1, wherein component (b) comprises at least one substance selected from polyacrylic acid salts, acrylate/C10-30 alkylacrylate crosspolymers and carrageenan in addition to one or more cosmetically active substances.

16. The product of claim 1, wherein one or more cosmetically active substances are comprised in a self-adhesive matrix of component (c).

17. The product of claim 16, wherein the self-adhesive matrix comprises at least one of a polyacrylate and a polyisobutylene in addition to the one or more cosmetically active substances.

18. The product of claim 1, wherein selected components are arranged adjacent to each other.

19. The product of claim 18, wherein selected components are present in a single package.

20. The product of claim 1, wherein component (a) is present in at least one of a blister package, a pan and a dispenser system.

21. The product of claim 1, wherein all of the selected components can be used for at least one identical purpose which is selected from a cosmetic treatment and a care and/or beautification of human skin.

22. A cosmetic combination product, wherein the product comprises at least two components selected from components (a) to (c):
(a) one or more dietary supplements which are present as at least one of a soft gelatin capsule, a hard gelatin capsule, a powder, a granulate, a globule, a lozenge, a tablet, an effervescent tablet, a sucking tablet, a coated tablet, a buccal tablet, a sublingual tablet, a bonbon, a chewing gum, a solution, a tincture, an emulsion, a juice, a concentrate, a syrup and a drinking ampoule,
(b) one or more cosmetic preparations for topical application to human skin which are present as at least one of an emulsion and a gel;
(c) one or more self-adhesive patches for adhesion to human skin,
and wherein each of the selected components (a) to (c) comprises at least one cosmetically active substance which is also comprised in the other selected component(s) and is selected from one or more of carnitine and derivatives thereof, tea extracts (green tea), mate tea (Ilex paraguariensis), white tea extracts (Camelia sinensis), herb tea extract, ubiquinone Q10, creatine, creatinine, taurine, caffeine, flavonoids, vitamin E, mate tea extract, conjugated linoleic acid (CLA) and derivatives thereof, safflower extract, pantothenic acid, B vitamins, biotin, amino acids and derivatives thereof and folic acid.

23. The product of claim 22, wherein the product comprises components (a), (b) and (c).

24. The product of claim 22, wherein the product comprises components (a) and (b), but no component (c).

25. The product of claim 22, wherein component (a) is present and comprises a soft gelatin capsule.

26. The product of claim 22, wherein the at least one cosmetically active substance comprises one or more substances selected from conjugated linoleic acid (CLA) and derivatives thereof, L-carnitine and derivatives thereof, vitamin E and mate tea extract.

27. The product of claim 25, wherein the at least one cosmetically active substance comprises at least one of L-carnitine and a derivative thereof.

28. The product of claim 22, wherein the at least one cosmetically active substance comprises at least one of conjugated linoleic acid (CLA) and a derivative thereof, at least one of L-carnitine and a derivative thereof, vitamin E and mate tea extract.

29. A method for the cosmetic prophylaxis or treatment of unevenness in the skin and/or for the treatment of cellulite, wherein the method comprises using the combination product of claim 1.

30. A process for making a combination product which is suitable for the care and/or beautification of human skin, wherein the process comprises providing a combination of at least two components selected from components (a) to (c):
(a) one or more dietary supplements,
(b) one or more cosmetic preparations for topical application to human skin, and
(c) one or more patches for adhesion to human skin,
wherein each of the selected components (a) to (c) comprises at least one cosmetically active substance which is also comprised in the other selected component(s).
